(19) United States
(12) Patent Application Publication
(10) Pub. No.: US 2026/0212476 A1
(43) Pub. Date: Jul. 23, 2026

(54) DETERMINATION DEVICE, DETERMINATION METHOD, AND PROGRAM

(71) Applicant: FUJIFILM Corporation, Tokyo (JP)

(72) Inventor: Yosuke NISHIURA, Kanagawa (JP)

(73) Assignee: FUJIFILM Corporation, Tokyo (JP)

(21) Appl. No.: 19/571,426

(22) Filed: Mar. 18, 2026

Related U.S. Application Data
(63) Continuation of application No. PCT/JP2024/030214, filed on Aug. 26, 2024.

(30) Foreign Application Priority Data
Sep. 22, 2023 (JP) 2023-158209

(51) Int. Cl. G06T 7/00; G01S 17/34; G06T 7/593; G06T 7/60

(52) U.S. Cl. CPC G06T 7/0002; G01S 17/34; G06T 7/593; G06T 7/60

(57) ABSTRACT

Provided are a determination device, a determination method, and a program capable of predicting a risk degree of a flaked-off object of a building before the flaked-off object falls. A determination device that determines a predicted risk degree of a flaked-off object, the determination device including a processor and a memory that stores a program to be executed by the processor, in which the processor is configured to acquire three-dimensional measurement data of a surface of a building measured by a measurement device, detect a bulging region, which is a precursor of the flaked-off object, based on the three-dimensional measurement data, estimate a size of the flaked-off object based on the bulging region, estimate a weight of the flaked-off object from the size and a cover thickness from the surface of the building to a reinforcing bar or a steel material, and determine the risk degree based on the weight.

### CROSS-REFERENCE TO RELATED APPLICATIONS

The present application is a Continuation of PCT International Application No. PCT/JP2024/030214 filed on Aug. 26, 2024 claiming priority under 35 U.S.C § 119(a) to Japanese Patent Application No. 2023-158209 filed on Sep. 22, 2023. Each of the above applications is hereby expressly incorporated by reference, in its entirety, into the present application.

### BACKGROUND OF THE INVENTION

### 1. Field of the Invention

The present invention relates to a determination device, a determination method, and a program, and particularly to a technique of determining a risk degree of a predicted flaked-off object.

### 2. Description of the Related Art

In the related art, a main inspection method for floating and peeling (flaking) of an infrastructure structure (such as a bridge or a tunnel, hereinafter referred to as a building) has been performed by an inspector going to a site and visually observing a surface state such as a crack or water leakage. The inspector taps a place determined to be a location with a high risk of floating and peeling with a hammer or a metal rod, and distinguishes a difference in sound between a normal portion and an abnormal portion to determine a deterioration degree.

On the other hand, various inspection techniques have been developed, and it is expected that the efficiency of inspection work will be improved by using these inspection techniques in the future. Various techniques have also been proposed for the inspection related to the floating and peeling of the building described above.

For example, in a flaking prediction diagnosis method described in JP2016-006398A, an infrared camera captures an infrared thermal image of a surface of a concrete building, an outside air temperature near the surface thereof is measured at the same time, a peeled portion temperature difference as a temperature difference between a sound portion and a peeled portion and a measurement temperature environment as a difference between a surface temperature of the sound portion and the outside air temperature are calculated based on the infrared thermal image and the outside air temperature, a temperature environment coefficient as a ratio of the calculated peeled portion temperature difference to the calculated measurement temperature environment is calculated, and a risk degree of flaking of cover concrete (concrete from reinforcing bar surface to concrete surface) is quantitatively evaluated according to the temperature environment coefficient. Further, a flaking risk degree calculated at a previous time is compared with a flaking risk degree calculated at a current time to predict a flaking timing.

### SUMMARY OF THE INVENTION

Although there is a proposal of a technique for predicting the flaking in the related art, as in the technique described in JP2016-006398A, there is no proposal of a technique for quantifying, for a portion predicted to be flaked off once, the risk degree in a case where the portion is flaked off.

The present invention has been made in view of such circumstances, and an object of the present invention is to provide a determination device, a determination method, and a program capable of predicting a risk degree of a flaked-off object of a building before the flaked-off object falls.

According to a first aspect of the present invention, there is provided a determination device that determines a predicted risk degree of a flaked-off object, the determination device including a processor and a memory that stores a program to be executed by the processor, in which the processor is configured to acquire three-dimensional measurement data of a surface of a building measured by a measurement device, detect a bulging region, which is a precursor of the flaked-off object, based on the three-dimensional measurement data, estimate a size of the flaked-off object based on the bulging region, estimate a weight of the flaked-off object from the size and a cover thickness from the surface of the building to a reinforcing bar or a steel material, and determine the risk degree based on the weight.

In the first aspect of the present invention, the three-dimensional measurement data of the surface of the building measured by the measurement device is acquired, the bulging region is detected based on the three-dimensional measurement data, the size of the flaked-off object is estimated, the weight of the flaked-off object is estimated, and the risk degree is determined. Accordingly, in the first aspect of the present invention, it is possible to predict the risk degree of the flaked-off object of the building before the flaked-off object falls.

According to a second aspect of the present invention, in the determination device according to the first aspect, the processor is configured to acquire information on a surface displacement from the three-dimensional measurement data to detect the bulging region based on the information on the surface displacement.

According to a third aspect of the present invention, in the determination device according to the first aspect, the processor is configured to acquire information on a surface displacement from the three-dimensional measurement data to create a contour diagram of the surface based on the information on the surface displacement, and detect a bulging of the surface based on the contour diagram to estimate the size based on the bulging.

According to a fourth aspect of the present invention, in the determination device according to the third aspect, the processor is configured to acquire crack information on the surface, and correct the size based on the crack information and the contour diagram.

According to a fifth aspect of the present invention, in the determination device according to the fourth aspect, the processor is configured to acquire an image of the surface, and acquire the crack information from the image.

According to a sixth aspect of the present invention, in the determination device according to the first aspect, the processor is configured to correct the determined risk degree based on the size.

According to a seventh aspect of the present invention, in the determination device according to the first aspect, the processor is configured to acquire information on a change over time of floating to update the size based on the information on the change over time of the floating.

According to an eighth aspect of the present invention, in the determination device according to the first aspect, the measurement device includes a LiDAR or a stereo camera.

According to a ninth aspect of the present invention, in the determination device according to the eighth aspect, the three-dimensional measurement data is measured by the LiDAR of a frequency modulated continuous wave (FMCW) type.

According to a tenth aspect of the present invention, in the determination device according to the first aspect, a material of the surface of the building includes concrete or a concrete repair material.

According to an eleventh aspect of the present invention, there is provided a determination method using a determination device that determines a predicted risk degree of a flaked-off object, in which the determination device includes a processor and a memory that stores a program to be executed by the processor, the determination method including, via the processor, a step of acquiring three-dimensional measurement data of a surface of a building measured by a measurement device, a step of detecting a bulging region, which is a precursor of the flaked-off object, based on the three-dimensional measurement data, a step of estimating a size of the flaked-off object based on the bulging region, a step of estimating a weight of the flaked-off object from the size and a cover thickness from the surface of the building to a reinforcing bar or a steel material, and a step of determining the risk degree based on the weight.

According to a twelfth aspect of the present invention, there is provided a program that executes a determination method using a determination device that determines a predicted risk degree of a flaked-off object, in which the determination device includes a processor and a memory that stores a program to be executed by the processor, the program causing the processor to execute a step of acquiring three-dimensional measurement data of a surface of a building measured by a measurement device, a step of detecting a bulging region, which is a precursor of the flaked-off object, based on the three-dimensional measurement data, a step of estimating a size of the flaked-off object based on the bulging region, a step of estimating a weight of the flaked-off object from the size and a cover thickness from the surface of the building to a reinforcing bar or a steel material, and a step of determining the risk degree based on the weight.

According to the present invention, it is possible to predict the risk degree of the flaked-off object of the building before the flaked-off object falls.

### BRIEF DESCRIPTION OF THE DRAWINGS

FIG. 1 is a graph showing a relationship between a lapse of time after construction of a building and a surface displacement of the building, and is a diagram showing an example of a cross section of the building at each time of inspection.

FIG. 2 is a schematic diagram of an inspection system of the building including a flaking prediction device according to the present invention.

FIG. 3 is an external view including an FMCW type LiDAR of one embodiment of a three-dimensional measurement device.

FIG. 4 is a diagram showing an embodiment in which a stereo camera measures a three-dimensional shape of a surface of the building.

FIG. 5 is a cross-sectional view of the vicinity of the surface of the building, which shows an example of a mechanism in which the surface of the building is peeled off.

FIG. 6 is a cross-sectional view of the vicinity of the surface of the building, which shows another example of the mechanism in which the surface of the building is peeled off.

FIG. 7 is a block diagram showing an embodiment of a hardware configuration of the flaking prediction device according to the present invention.

FIG. 8 is a block diagram showing functions realized by a processor executing a dedicated program.

FIG. 9 is a flowchart for describing a determination method using a determination device.

FIGS. 10A, 10B and 10C are diagrams for describing three-dimensional measurement data and a contour diagram.

FIGS. 11A, 11B and 11C are diagrams for describing three-dimensional measurement data and a contour diagram.

FIGS. 12A, 12B and 12C are diagrams for describing three-dimensional measurement data and a contour diagram.

FIG. 13 is a diagram for describing a detection step and a size estimation step.

FIG. 14 is a diagram for describing the detection step and the size estimation step.

FIG. 15 is a diagram showing a change over time of an impact load.

FIG. 16 is a block diagram showing the functions realized by the processor.

FIG. 17 is a diagram for describing a case where a bulging region is corrected based on crack information.

FIG. 18 is a block diagram showing functions realized by the processor.

FIG. 19 is a diagram showing an example of information on a change over time of floating.

### DESCRIPTION OF THE PREFERRED EMBODIMENTS

Hereinafter, preferred embodiments of a determination device, a determination method, and a program according to the present invention will be described with reference to accompanying drawings.

### <Detection of Flaked-Off Object>

First, detection of a flaked-off object will be described. The flaked-off object is a falling object that falls due to peeling or flaking of concrete on a surface of a building. The flaked-off object is generated from a ceiling, a wall, a pillar, a bridge pier, or the like of the building due to aging deterioration or the like.

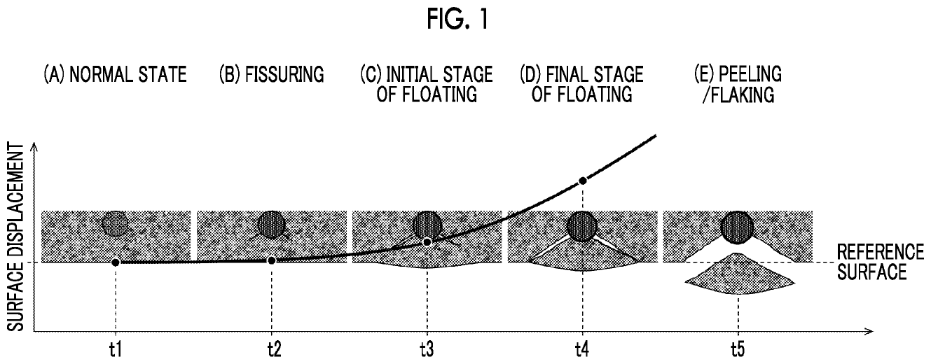

FIG. 1 is a graph showing a relationship between a lapse of time after construction of a building and a surface displacement of the building, and is a diagram showing an example of a cross section of the building at each time of inspection.

In FIG. 1, the displacement of the surface of the building is measured at an inspection start point in time (measurement start point in time at a time of construction) t1 of the building and at each inspection point in time (t2, t3, t4, t5, . . . ) after the measurement start point in time. With a comparison between the displacements at the same position on the surface of the building, it is possible to observe a location where the surface bulges with a lapse of time from the construction of the building.

In the example shown in FIG. 1, the building at the measurement start point in time t1 is in an (A) normal state, but the surface of the building slightly bulges due to a deterioration phenomenon ((B) fissuring) of the building at the inspection point in time t2. The bulging at this timing is not able to be checked by visual observation or the like. The “fissuring” is often caused by corrosion and thickening of a steel material (reinforcing bar) inside the building.

In a (C) initial stage of floating shown at the inspection point in time t3, the “fissuring” also progresses as the corrosion of the reinforcing bar progresses, and the surface of the building bulges (“floating” occurs).

In a (D) final stage of floating shown at the inspection point in time t4, the “fissuring” further progresses, reaches the surface of the building, and the “floating” also further increases.

The inspection point in time t5 indicates a point in time at which cover concrete (concrete from reinforcing bar surface to concrete surface) falls ((E) peeling/flaking).

In the example shown in FIG. 1, it can be seen that the displacement (bulging amount) of the surface of the building, which is measured for each inspection, gradually increases and the cover concrete peels off and flakes off.

In addition to the deterioration of the reinforcing bar due to the corrosion of the reinforcing bar, the deterioration phenomenon of the building includes deterioration of concrete strength and concrete deterioration such as the fissuring and surface deterioration. Since the surface of the building bulges in all the deterioration phenomena of the building, it is possible to predict a peeling/flaking timing, regardless of the cause of deterioration, from a change over time of the bulging amount of the surface.

### <Outline Configuration of Inspection System>

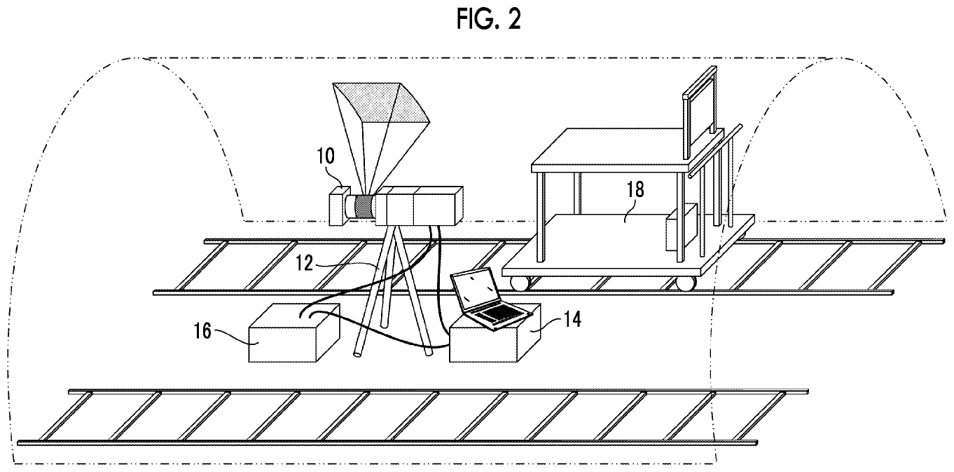

FIG. 2 is a schematic diagram of an inspection system of a building including a determination device.

The inspection system shown in FIG. 2 is to inspect a tunnel of a railroad, and comprises a three-dimensional measurement device (measurement device) 10, a data processing device 14, and a power supply device 16.

The three-dimensional measurement device 10 is mounted on a tripod 12, but may be mounted on a carriage 18 that travels on a railroad track.

The three-dimensional measurement device 10 is a light detection and ranging (LiDAR) in the present example, and is particularly a frequency modulated continuous wave (FMCW) type LiDAR capable of performing distance measurement in an order of several hundred μm. However, the present invention is not limited to a case where distance measurement data (three-dimensional measurement data) measured by the FMCW type LiDAR is used.

### <Three-Dimensional Measurement Device>

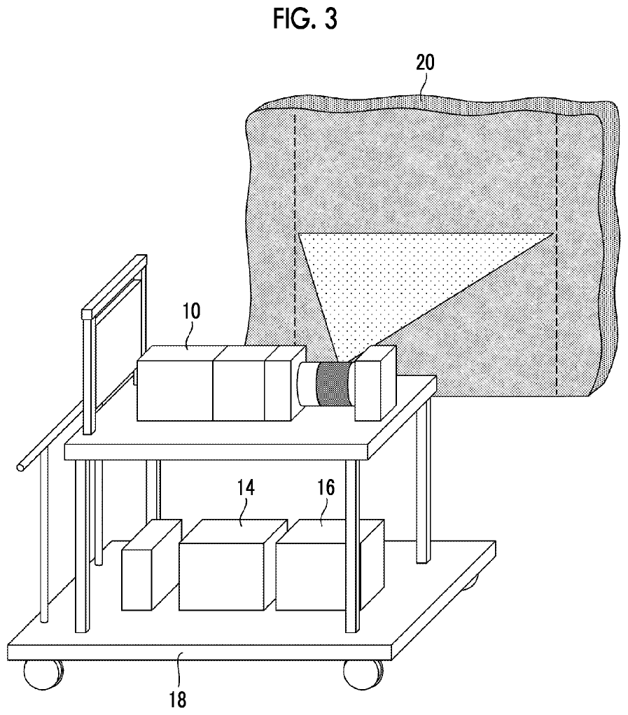

FIG. 3 is an external view including the FMCW type LiDAR of one embodiment of the three-dimensional measurement device 10.

In FIG. 3, the three-dimensional measurement device 10 is mounted on the carriage 18 that travels on the railroad track as shown in FIG. 2 to measure a distance to a surface of the tunnel, which is a railroad building.

The carriage 18 is mounted with the data processing device 14 and the power supply device 16, in addition to the three-dimensional measurement device 10. The power supply device 16 supplies power to the three-dimensional measurement device 10 and the data processing device 14.

The three-dimensional measurement device 10 measures a distance to a wall surface (surface) 20 of the tunnel to acquire the three-dimensional measurement data indicating a shape of the wall surface 20 of the tunnel.

In the example shown in FIG. 3, the three-dimensional measurement device 10 scans the wall surface 20 shown in FIG. 3 in a left-right direction (main scanning direction) at a high speed with laser light of the FMCW type, and causes a scanning line to move in an up-down direction (sub-scanning direction) of the wall surface 20 to perform the scanning. Accordingly, the distance measurement is performed from a measurement head of the three-dimensional measurement device 10 to a large number of measurement points on each scanning line of the laser light. Three-dimensional measurement data of a polar coordinate system, which consists of an irradiation direction of the laser light and a measured distance, is converted into three-dimensional measurement data of a rectangular coordinate system to acquire the three-dimensional measurement data indicating the shape of the wall surface 20. In the present example, three-dimensional measurement data (point group data) of a large number of measurement points is acquired as the three-dimensional measurement data.

It is considered that the three-dimensional measurement device 10 performs measurement of a minute uneven shape of the wall surface 20 under the following conditions.
Measurement accuracy: 50 μm
Measurement distance: 2 m to 7 m
Measurement speed: 10 m²/sec in terms of area (speed of laser light is equivalent to 4000 rpm)

Further, the three-dimensional measurement device 10 acquires, for example, the three-dimensional measurement data of the wall surface 20 at a constant interval during the movement of the carriage 18. However, the three-dimensional measurement data is preferably acquired such that measurement regions of the three-dimensional measurement data acquired at each interval partially overlap. This is for panorama composition of the three-dimensional measurement data acquired at each interval.

The three-dimensional measurement device 10 can achieve the measurement accuracy and the like described above with the use of the FMCW type LiDAR, but the conditions such as the measurement accuracy of the three-dimensional measurement data required in the present invention are not limited to the above example. Further, the three-dimensional measurement device 10 is not limited to the FMCW type LiDAR, and various types of LiDAR can be employed.

For example, a time of flight (TOF) type LiDAR that measures a flight time of pulse-projected light to measure the distance to the wall surface 20 can be used instead of the FMCW type LiDAR. Further, the three-dimensional shape of the wall surface 20 can be measured by a stereo camera.

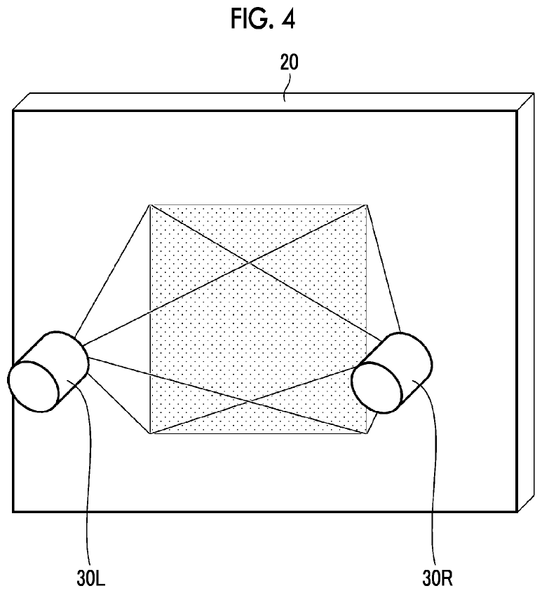

FIG. 4 is a diagram showing an embodiment in which a stereo camera measures a three-dimensional shape of the surface of the building.

The stereo camera shown in FIG. 4 consists of a left camera 30L and a right camera 30R, and measures the distance to the wall surface 20 of an imaging target by a triangulation method.

In addition, various three-dimensional measurement devices 10 can be employed as the three-dimensional measurement device 10 that measures the distance to the wall surface 20 (that is, three-dimensional measurement data of wall surface), such as a laser radar three-dimensional shape measurement device described in JP1997-297014A (JP-H9-297014A), a measurement device by an optical cutting method using an imaging device and a slit laser light projector described in JP2016-31249A, an FM laser radar type distance measurement device described in JP3194586B, and an optical distance meter described in JP3583906B.

The three-dimensional shape of the wall surface 20 of the tunnel is measured by the three-dimensional measurement device 10 at a time of measurement start (construction) of the tunnel and at a time of regular inspection after the construction. The measured three-dimensional measurement data of the wall surface is stored in a storage device in the data processing device 14 or an external storage device at the time of measurement start and at the time of regular inspection.

### <Mechanism of Peeling of Surface Material of Building>

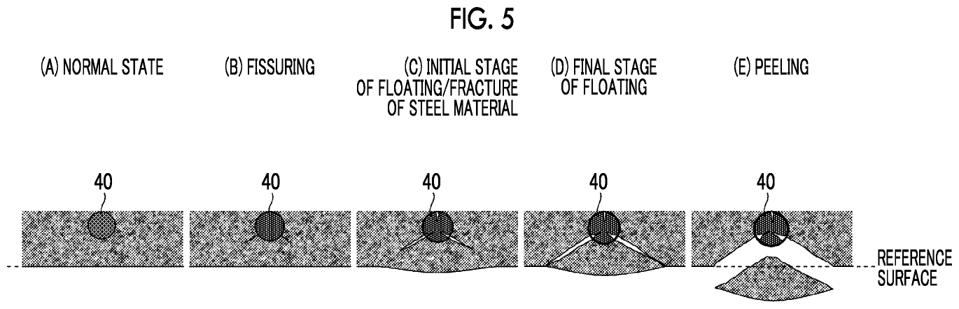

FIG. 5 is a cross-sectional view of the vicinity of the surface of the building, which shows an example of a mechanism in which the surface of the building is peeled off.

An “(A) normal state” of FIG. 5 refers to a state of being normal, for example, at the time of construction of the building. The surface in this state is defined as a reference surface. In FIG. 5, 40 is a steel material (reinforcing bar).

The “(B) fissuring”, “(C) initial stage of floating/fracture of steel material”, “(D) final stage of floating”, and “(E) peeling” of FIG. 5 occur due to corrosion (for example, salt damage or water leakage) of the reinforcing bar 40 or the like, and occur according to the number of years elapsed since the time of construction of the tunnel.

In “(C) Initial stage of floating/fracture of steel material” of FIG. 5 and subsequent stages, the surface of the building is gradually higher than the reference surface (“floating” occurs), and the cover concrete is peeled off.

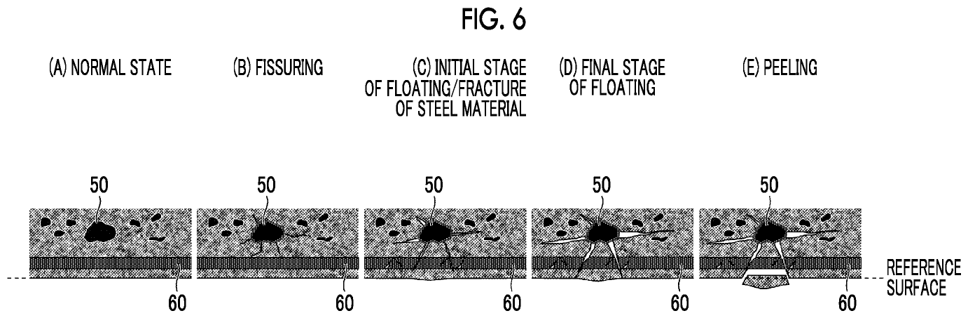

FIG. 6 is a cross-sectional view of the vicinity of the surface of the building, which shows another example of the mechanism in which the surface of the building is peeled off.

An “(A) normal state” of FIG. 6 refers to a state of being normal, for example, at the time of construction of the building. The surface in this state is defined as a reference surface. In FIG. 6, 50 indicates a reactive aggregate, and 60 indicates a steel material.

The “(B) fissuring”, “(C) initial stage of floating/fracture of steel material”, “(D) final stage of floating”, and “(E) peeling” of FIG. 6 occur due to the deterioration of concrete strength (for example, alkali reaction of the reactive aggregate 50) or the like, and occur according to the number of years elapsed since the time of construction.

In “(C) Initial stage of floating/fracture of steel material” of FIG. 6 and subsequent stages, the surface of the building is gradually higher than the reference surface (“floating” occurs), and the cover concrete from a surface of the steel material 60 to the surface is peeled off.

As shown in FIG. 5 and FIG. 6, in a case where “floating” occurs due to the change over time in the surface of the building, “peeling” occurs in the future. This applies to all cases regardless of the cause of “floating”. That is, the presence or absence, material, and shape of the reinforcing bar, a material and shape of the concrete, an insertion method of the reinforcing bar into the concrete, a construction method, and a cause of corrosion (neutralization, frost damage, construction defects, or the like) are not taken into account.

### <Hardware Configuration of Determination Device>

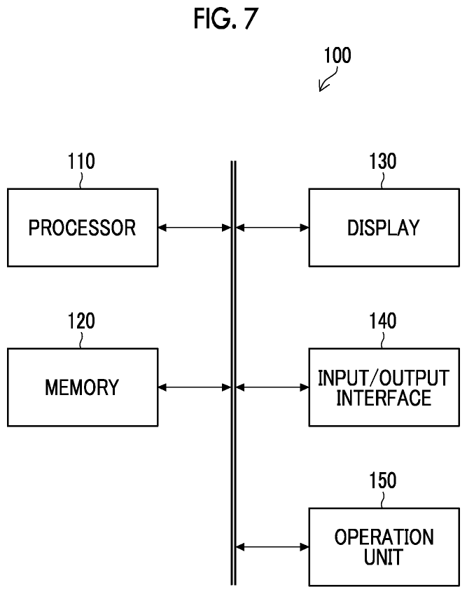

FIG. 7 is a block diagram showing an embodiment of a hardware configuration of the determination device according to the embodiment of the present invention.

A determination device 100 shown in FIG. 7 is configured of, for example, a personal computer, a workstation, or the like, and comprises a processor 110, a memory 120, a display 130, an input/output interface 140, and an operation unit 150. The determination device 100 can be incorporated as one function of the data processing device 14 shown in FIG. 2.

The processor 110 is configured of a central processing unit (CPU) and the like, and integrally controls each unit of the determination device 100 and executes a program for performing the determination method to execute various types of processing for predicting the flaking of the surface of the building. Details of various types of processing performed by the processor 110 will be described below.

The memory 120 includes a flash memory, a read-only memory (ROM), a random access memory (RAM), a hard disk device, and the like. The flash memory, the ROM, or the hard disk device is a non-volatile memory that stores an operating system, various programs including a program for causing the determination method according to the embodiment of the present invention to be executed, and the like. Further, the non-volatile memory (storage device), such as the flash memory and the hard disk device, stores the three-dimensional measurement data of the surface of the building, which is measured by the three-dimensional measurement device 10 at the time of measurement start of the building and at the time of regular inspection, together with a measurement point in time.

The RAM functions as a work area for the processing by the processor 110. Further, various programs stored in the flash memory or the like, the three-dimensional measurement data of the surface of the building, and the like are temporarily stored. It should be noted that a part (RAM) of the memory 120 may be built into the processor 110.

The display 130 displays a screen for operation of the determination device 100, displays a graph created by the determination device 100, or displays a surface property image or the like of the building, and is also used as a part of a graphical user interface (GUI) in a case where a user input of a point of interest on the surface of the building is received from the operation unit 150.

The input/output interface 140 includes a connection unit that is connectable to an external device, a communication unit that is connectable to a network, and the like. A universal serial bus (USB), a high-definition multimedia interface (HDMI) (HDMI is a registered trademark), or the like can be employed as the connection unit that is connectable to the external device.

The determination device 100 can be configured as a device independent of the data processing device 14. In this case, the processor 110 can acquire the three-dimensional measurement data of the surface of the building from the data processing device 14 via the input/output interface 140. Alternatively, in a case where the three-dimensional measurement data is stored in a cloud, the three-dimensional measurement data of the surface of the building can be acquired from the cloud via the input/output interface 140. Further, the processor 110 can store the three-dimensional measurement data acquired in this manner in the memory 120.

The operation unit 150 includes a pointing device such as a mouse, a keyboard, and the like, and uses a display screen of the display 130 to function as a part of the GUI that receives an instruction input by a user operation.

### First Embodiment

A first embodiment of the present invention will be described.

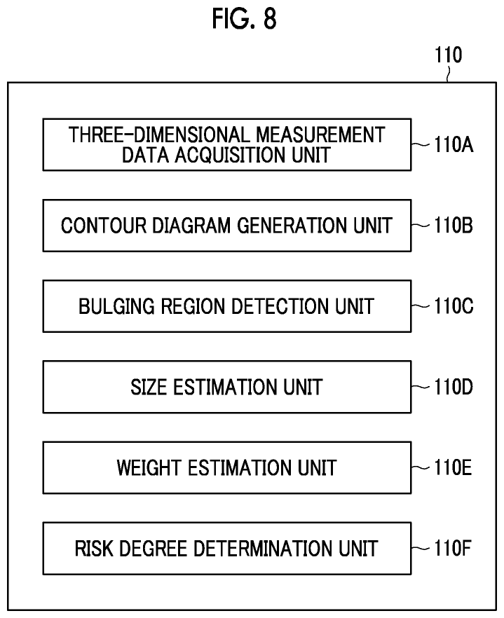

FIG. 8 is a block diagram showing functions realized by the processor 110 executing a dedicated program.

The processor 110 realizes a three-dimensional measurement data acquisition unit 110A, a contour diagram generation unit 110B, a bulging region detection unit 110C, a size estimation unit 110D, a weight estimation unit 110E, and a risk degree determination unit 110F.

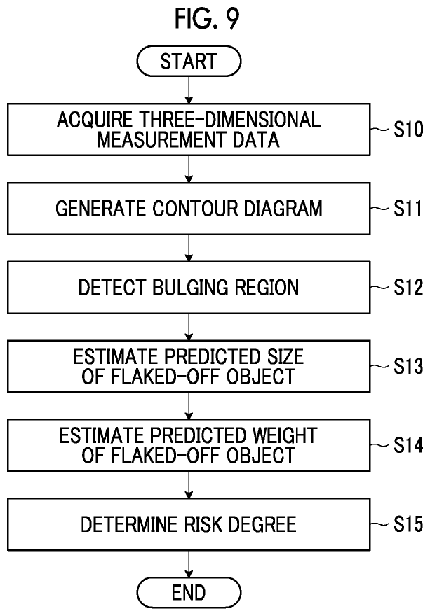

FIG. 9 is a flowchart for describing the determination method using the determination device 100. The determination method is performed by the processor 110 of the determination device 100 executing the dedicated program. Each step described below is performed in each functional block diagram of the processor 110 described above.

The three-dimensional measurement data acquisition unit 110A acquires the three-dimensional measurement data of the surface of the building acquired by the three-dimensional measurement device 10 (step S10: three-dimensional measurement data acquisition step). Thereafter, the contour diagram generation unit 110B generates a contour diagram based on the acquired three-dimensional measurement data (step S11: contour diagram generation step). Next, the bulging region detection unit 110C detects a bulging region, which is a precursor of the flaked-off object, based on the contour diagram (step S12: detection step). Next, the size estimation unit 110D estimates a predicted size (area) of the flaked-off object based on the bulging region (step S13: size estimation step). Thereafter, a weight is estimated from the size estimated by the size estimation unit 110D and a cover thickness (step S14: weight estimation step). Thereafter, the risk degree in a case where the flaked-off object falls is determined based on the estimated weight (step S15: risk degree determination step).

Hereinafter, each step will be described in detail.

### <Three-Dimensional Measurement Data Acquisition Step and Contour Diagram Generation Step>

First, the three-dimensional measurement data acquisition step and the contour diagram generation step will be described. The three-dimensional measurement data acquisition step is performed by the three-dimensional measurement data acquisition unit 110A, and the contour diagram generation step is performed by the contour diagram generation unit 110B.

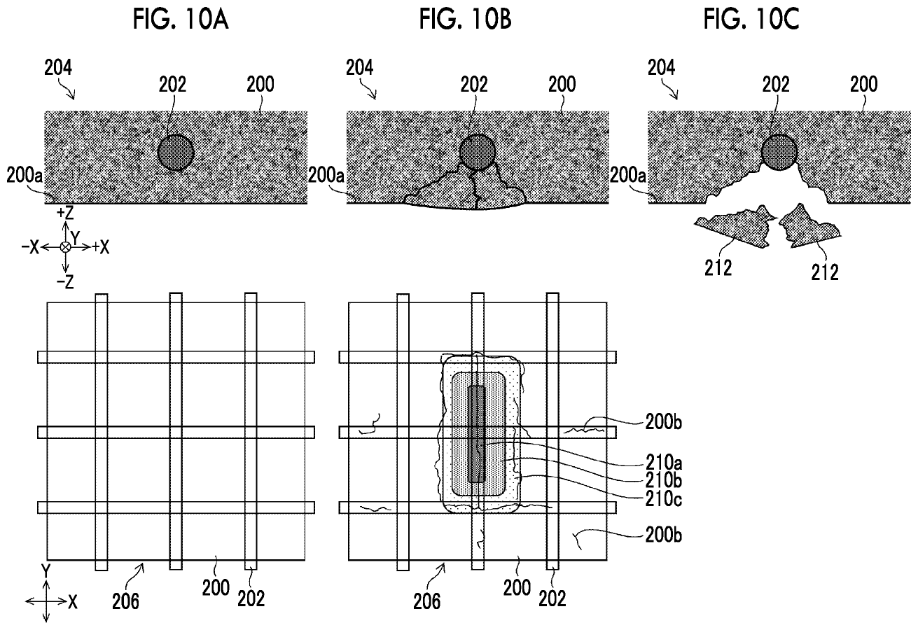

FIGS. 10 to 12 are diagrams for describing the three-dimensional measurement data and the contour diagram. FIGS. 10 to 12 show a model diagram 204 and a contour diagram 206 in a case where a change is made from a normal state ((A) described in FIG. 1) to floating ((C) or (D) described in FIG. 1) and occurrence of flaked-off object ((E) described in FIG. 1). The model diagram 204 schematically illustrates a reinforcing bar 202 and a concrete 200, and has a surface 200a of the concrete 200 on an X-Y plane, which is not shown. Further, the contour diagram 206 shows a surface displacement of the surface 200a on the X-Y plane of the model diagram 204, and illustrates the bulging from the reference surface as described in FIG. 1.

FIGS. 10A, 10B and 10C are diagrams for describing Specific Example 1 of the surface displacement of the building.

FIG. 10A shows the model diagram 204 and the contour diagram 206 of a part of the building in the normal state in which the floating has not occurred. As shown in the corresponding contour diagram 206, the bulging from the reference surface is not detected on the surface 200a in a case of the normal state.

FIG. 10B shows the model diagram 204 and the contour diagram 206 of a part of the building in which the floating has occurred on the surface 200a. In a case where the floating occurs on the surface 200a, a region that bulges from the reference surface (bulging region) is shown in the contour diagram 206. Specifically, a bulging region 210a, a bulging region 210b, and a bulging region 210c are shown, in the contour diagram 206, in descending order of the surface displacement from the reference surface. Further, a crack 200b generated on the surface 200a is also depicted in the contour diagram 206.

FIG. 10C shows the model diagram 204 showing a case where the flaked-off object is generated. In the case shown in FIG. 10C, a state is shown in which a flaked-off object 212 is generated due to the floating shown in FIG. 10B. In this manner, the floating occurs, and as the floating increases, a part of the concrete between the reinforcing bar 202 and the surface 200a falls as the flaked-off object 212.

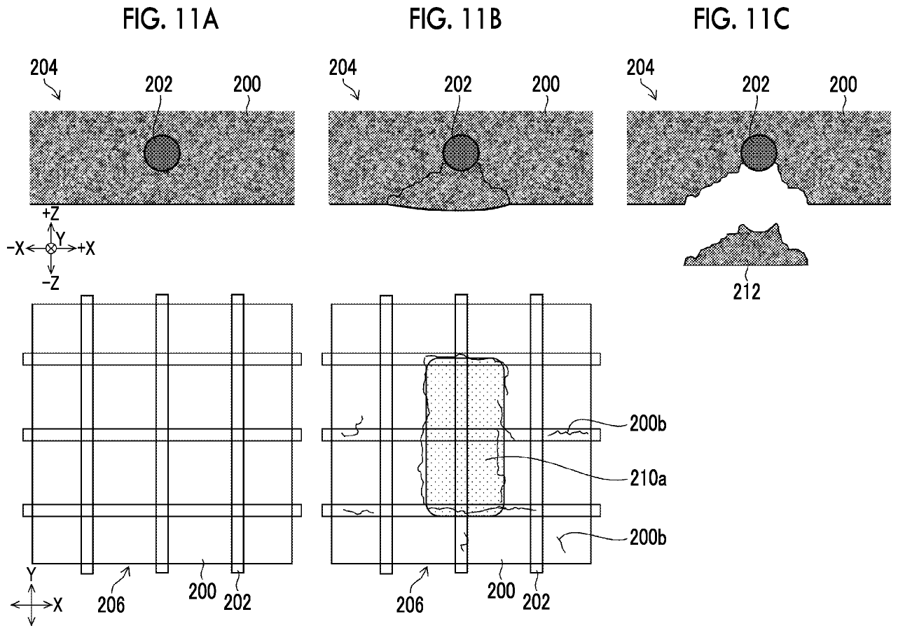

FIGS. 11A, 11B and 11C are diagrams for describing Specific Example 2 of the surface displacement of the building.

FIG. 11A shows the model diagram 204 and the contour diagram 206 of a part of the building in the normal state in which the floating has not occurred. As shown in the contour diagram 206, the bulging from the reference surface is not detected on the surface 200a in a case of the normal state.

FIG. 11B shows the model diagram 204 and the contour diagram 206 of a part of the building in which the floating has occurred on the surface 200a. In a case where the floating occurs on the surface 200a, the region that bulges from the reference surface (bulging region 210a) is shown in the contour diagram 206. Further, a crack 200b generated on the surface 200a is also depicted in the contour diagram 206.

FIG. 11C shows the model diagram 204 showing a case where the flaked-off object is generated. In the case shown in FIG. 11C, a state is shown in which the flaked-off object 212 is generated due to the floating shown in FIG. 11B. In this manner, the floating occurs, and as the floating increases, a part of the concrete between the reinforcing bar 202 and the surface 200a falls as the flaked-off object 212.

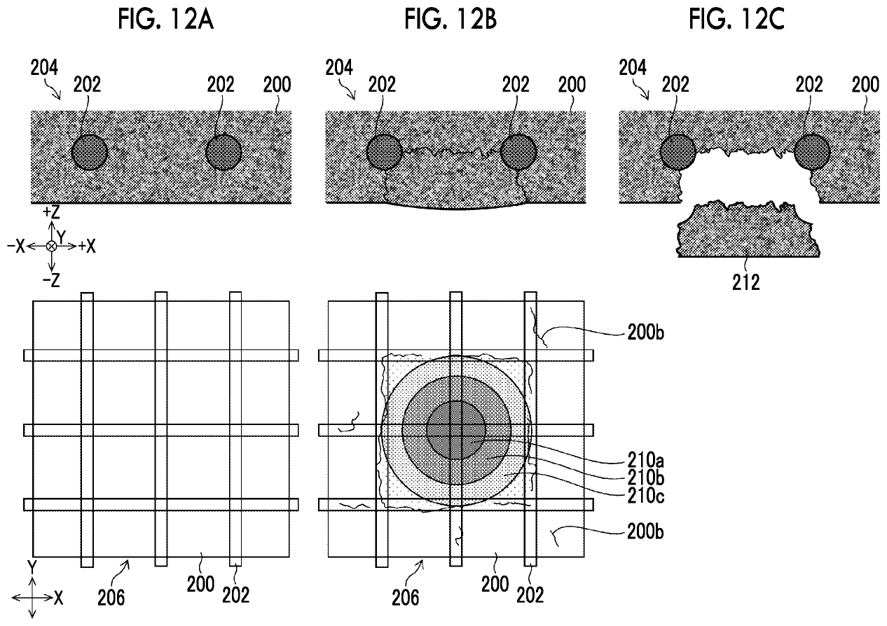

FIGS. 12A, 12B and 12C are diagrams for describing Specific Example 3 of the surface displacement of the building.

FIG. 12A shows the model diagram 204 and the contour diagram 206 of a part of the building in the normal state in which the floating has not occurred. As shown in the contour diagram 206, the bulging from the reference surface is not detected on the surface 200a in a case of the normal state.

FIG. 12B shows the model diagram 204 and the contour diagram 206 of a part of the building in which the floating has occurred on the surface 200a. In a case where the floating occurs on the surface 200a, a region that bulges from the reference surface (bulging region) is shown in the contour diagram 206. Specifically, a bulging region 210a, a bulging region 210b, and a bulging region 210c are shown, in the contour diagram 206, in descending order of the surface displacement from the reference surface. Further, a crack 200b generated on the surface 200a is also depicted in the contour diagram 206.

FIG. 12C shows the model diagram 204 showing a case where the flaked-off object is generated. In the case shown in FIG. 12C, a state is shown in which the flaked-off object 212 is generated due to the floating shown in FIG. 12B. In this manner, the floating occurs, and as the floating increases, a part of the concrete between the reinforcing bar 202 and the surface 200a falls as the flaked-off object 212.

As described above, the three-dimensional measurement data acquisition unit 110A acquires the three-dimensional measurement data from the three-dimensional measurement device 10, and the contour diagram generation unit 110B generates the contour diagram based on the acquired three-dimensional measurement data. With the generation of the contour diagram 206 in the contour diagram generation unit 110B, it is possible to accurately detect the bulging region. In the above description, a case has been described in which the contour diagram generation unit 110B generates the contour diagram, but the application of the present invention is not limited thereto. In the present invention, the three-dimensional measurement data acquisition unit 110A may directly detect the bulging region from the three-dimensional measurement data.

### <Detection Step, Size Estimation Step, and Weight Estimation Step>

Next, the detection step, the size estimation step, and the weight estimation step will be described. The detection step is performed by the bulging region detection unit 110C, the size estimation step is performed by the size estimation unit 110D, and the weight estimation step is performed by the weight estimation unit 110E.

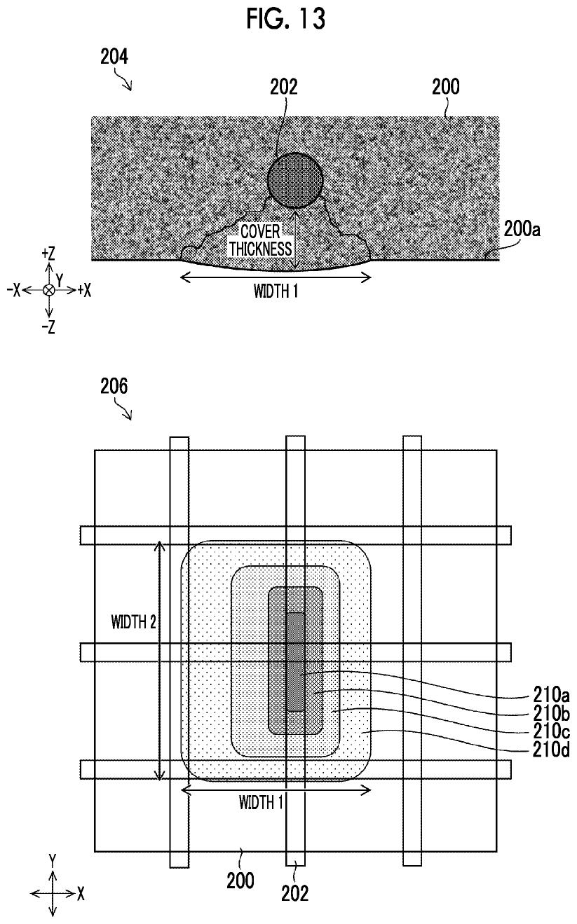

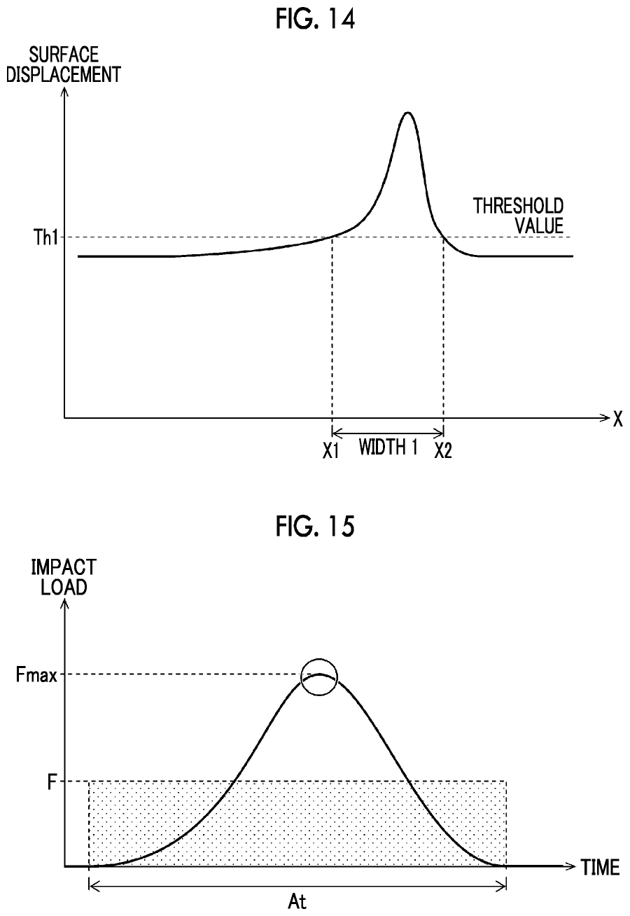

FIGS. 13 and 14 are diagrams for describing the detection step and the size estimation step.

FIG. 13 shows the model diagram and the contour diagram in a case where the floating occurs.

The bulging region detection unit 110C detects, as the bulging regions (bulging regions 210a to 210d), regions in which the bulging equal to or larger than a predetermined threshold value (for example, 0.5 mm) is detected from the contour diagram 206 of the surface displacement of the floating.

FIG. 14 is a diagram showing a surface displacement V in an X-axis direction. In FIG. 14, a horizontal axis indicates a distance (position) in the X-axis direction, and a vertical axis indicates the surface displacement V of the surface 200a. Further, a dotted line in the drawing indicates a threshold value Th1 of the surface displacement. A width 1 is acquired according to positions (X1 and X2 in the drawing) exceeding the threshold value Th1 in the surface displacement V. The bulging region detection unit 110C detects widths of the region having the bulging equal to or larger than the threshold value in the X-axis and a Y-axis. In this manner, the bulging region detection unit 110C acquires the width 1 of the bulging region in the X-axis direction and a width 2 of the bulging region in the Y-axis direction. The size estimation unit 110D acquires a product of the width 1 and the width 2 to calculate and estimate a size (area) of the bulging region. There may be various shapes of the bulging regions, and a method of calculating the area in accordance with the shape may be employed.

The weight estimation unit 110E estimates the weight from the estimated size and the cover thickness from the surface 200a of the building to the reinforcing bar 202 (or steel material (not shown)).

In the case shown in FIG. 13, the weight estimation unit 110E can calculate the weight by the following Equation (1).

$$\begin{matrix}[ Formula 1 ] & \\ Weight of flaked - off object = density of concrete \times width 1 \times cover thickness \times 1 / 2 \times width 2 & ( Equation 1 )\end{matrix}$$

The density of the concrete can be acquired based on a recipe of the concrete described in a design drawing or the like. Further, a sample may be collected in an actual site and measured to acquire the density of the concrete.

Further, the cover thickness can be acquired from the design drawing. Further, the surface of the concrete may actually be chipped to measure a distance to the reinforcing bar (or steel material) and thus to acquire the cover thickness, or an electromagnetic wave radar method or an electromagnetic induction method may be used to measure the cover thickness.

As described above, the bulging region detection unit 110C detects the bulging region, which is a precursor of the flaked-off object, based on the information (for example, contour diagram) of the surface displacement, and the size is estimated from the bulging region, and the weight of the flaked-off object is estimated from the size and the cover thickness. Accordingly, it is possible to accurately estimate the weight of a predicted flaked-off object that has not yet fallen.

### <Risk Degree Determination Step>

Next, the risk degree determination step will be described. The risk degree determination step is performed by the risk degree determination unit 110F. The risk degree determination unit 110F determines the risk degree based on the estimated weight of the flaked-off object.

For example, the risk degree determination unit 110F can quantitatively determine the risk degree by an impact load F calculated by the following Equation (2), based on the estimated weight of the flaked-off object.

$$\begin{matrix}[ Formula 2 ] & \\ F = M \cdot \sqrt{\frac{2 gH}{Δ t}} & ( 2 )\end{matrix}$$

Here, M is “estimated weight of flaked-off object”, Δt is “collision time”, and His “fall height”, and known numerical values are input for Δt and H.

The impact load F is an average of the impact load, and generally, the impact is changed over time, and Fmax is calculated by Gaussian fitting or the like to calculate a maximum impact load corresponding to the risk degree, as shown in FIG. 15. Regarding the determination of the risk degree using Equation (2) described above, a document is also referred to: “Experimental Study on Determination of RiskDegree in Case of Flaking Concrete Collision” [online] [searched on Aug. 29, 2023] (Internet: http://library.jsce.or.jp/jsce/open/00035/2015/70-05/70-05-0415.pdf) of the 70th Annual Academic Lecture Meeting of the Japan Society of Civil Engineers (September 2015).

Further, for example, the risk degree determination unit 110F may use a head injury criterion (HIC) calculated by the following Equation (3), which is widely known for evaluating the possibility of a head injury in a dummy test of an automobile, as the risk degree.

$$\begin{matrix}[ Formula 3 ] & \\ HIC = {( \frac{{\int }_{{t}_{1}}^{{t}_{2}} adt}{{t}_{2} - {t}_{1}} )}^{2.5} ( {t}_{2} - {t}_{1} ) & ( 3 )\end{matrix}$$

Here, a is “acceleration (unit: gravitational acceleration g) calculated based on weight of flaked-off object”, t₁ is “optional time point of pulse”, and t₂ is “time point at which HIC is maximized in a case where t₁ is decided”.

Regarding the determination of the risk degree using Equation (3) described above, a document is also referred to: “Basic Research on Safety Evaluation of Ceiling Materials Using Human Body Resistance Index, Part 3, Ceiling Material Falling Experiment 2” [online] [searched on Aug. 29, 2023] (Internet: http://space.iis.u-tokyo.ac.jp/main/report/20441.pdf) of the summary of academic lectures of the annual meeting of the Architectural Institute of Japan (Hokuriku) in September 2010.

As described above, it is possible to determine the risk degree by the risk degree determination unit 110F based on the weight of the predicted flaked-off object.

### Second Embodiment

Next, a second embodiment of the present invention will be described. In the present embodiment, the crack information is acquired, the crack information is reflected in the contour diagram, and the estimated size (area) of the flaked-off object is corrected.

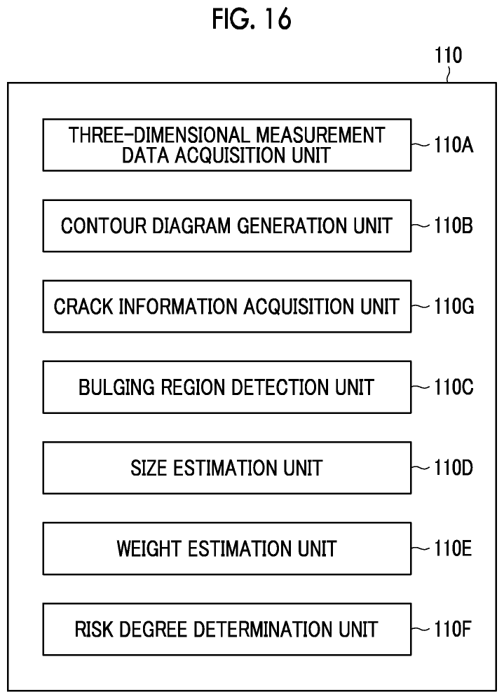

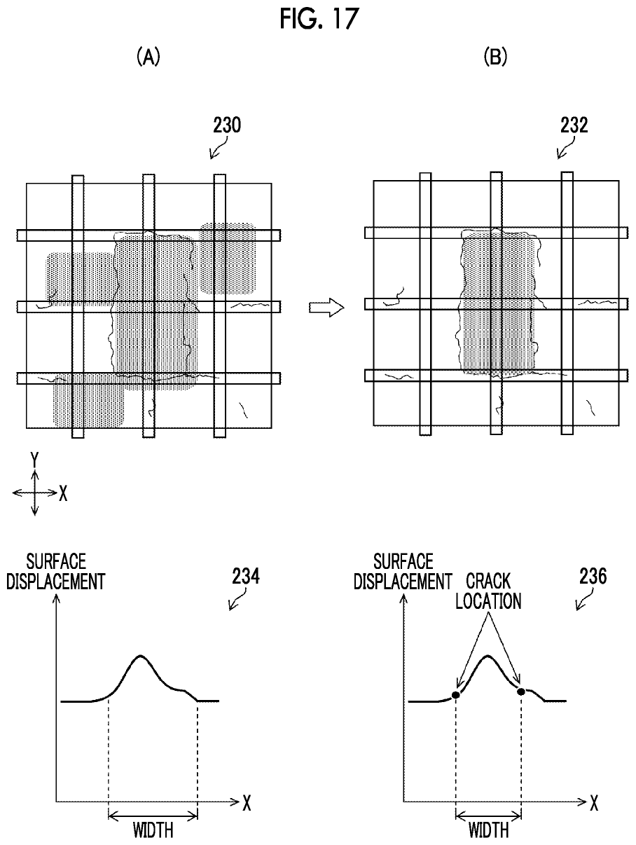

FIG. 16 is a block diagram showing functions realized by the processor 110 in the present embodiment. The same reference numerals will be assigned to the parts already described, and the description thereof will be omitted.

The processor 110 realizes the three-dimensional measurement data acquisition unit 110A, the contour diagram generation unit 110B, a crack information acquisition unit 110G, the bulging region detection unit 110C, the size estimation unit 110D, the weight estimation unit 110E, and the risk degree determination unit 110F.

For example, in a place where a large amount of floating has occurred, a place where a large amount of bulging, which is not floating, has occurred due to defects during construction, or the like, it may be difficult to specify the bulging region caused by the floating only with the contour diagram. In such a case, the crack information is acquired, the crack information is superimposed on the contour diagram to specify a position where the crack is present, and the size of the bulging region is corrected based on the position of the crack. The crack information acquisition unit 110G acquires an image of the surface of the building as the crack information to detect the crack based on the acquired image.

FIG. 17 is a diagram for describing a case where the bulging region is corrected based on the crack information.

(A) of FIG. 17 shows a contour diagram 230 and a diagram 234 showing the surface displacement in the X-axis direction before correction, and (B) of FIG. 17 shows a contour diagram 232 and a diagram 236 showing the surface displacement in the X-axis direction after correction.

Before the correction is performed based on the crack information, the detected bulging regions are scattered in various places, as shown in the contour diagram 230, and thus the bulging region based on the floating cannot be specified. As shown in FIG. 17, in a case where the bulging region is detected based on the surface displacement, the bulging regions are detected in a scattered manner. On the other hand, in a case where the correction is performed based on the crack information, the bulging region corresponding to the floating can be specified from the crack information, as shown in the contour diagram 232. Specifically, as shown in the diagrams 234 and 236 showing the surface displacement in the X-axis direction, the width of the bulging region is corrected to be smaller according to a location of the crack based on the crack information. For example, in this correction, a location where the surface displacement changes with a crack location interposed therebetween is set as a boundary of the bulging region to specify the width of the bulging region.

As described above, the crack information acquisition unit 110G acquires, for example, the image of the surface of the building as the crack information to detect the crack from the image. The crack information is superimposed on the contour diagram to correct the size estimated by the size estimation unit 110D. Accordingly, in the present embodiment, it is possible to estimate the size of the flaked-off object with high accuracy.

### Third Embodiment

Next, a third embodiment of the present invention will be described. In the present embodiment, information on the change over time of the floating is acquired, and the size is updated based on the information on the change over time of the floating. Here, the update of the size means estimating how the size, which is once estimated, will change in a case where a predetermined period has elapsed based on the information on the change over time of the floating.

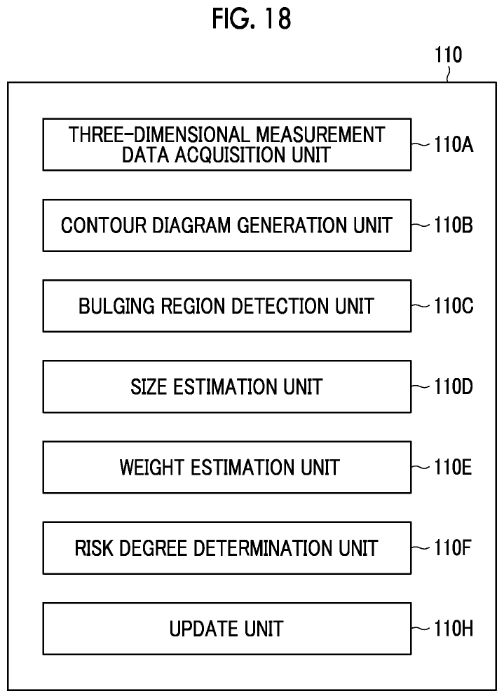

FIG. 18 is a block diagram showing functions realized by the processor 110 in the present embodiment. The same reference numerals will be assigned to the parts already described, and the description thereof will be omitted.

The processor 110 realizes the three-dimensional measurement data acquisition unit 110A, the contour diagram generation unit 110B, the bulging region detection unit 110C, the size estimation unit 110D, the weight estimation unit 110E, the risk degree determination unit 110F, and an update unit 110H.

The update unit 110H acquires the information on the change over time of the floating, and updates the size and the weight based on the information on the change over time of the floating.

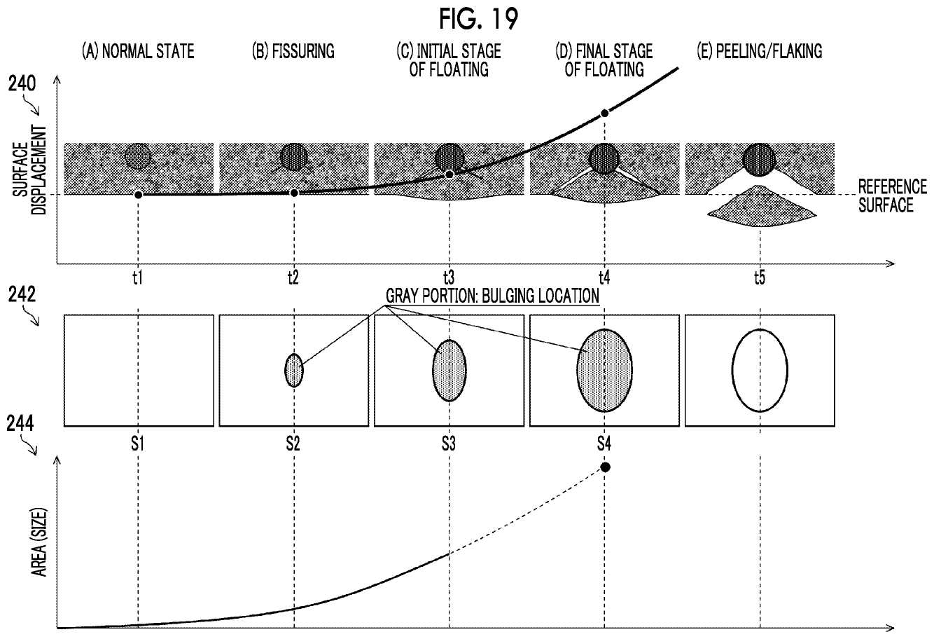

FIG. 19 is a diagram showing an example of the information on the change over time of the floating.

At a reference numeral 240, a graph is shown in which a vertical axis indicates a surface displacement and a horizontal axis indicates an elapsed time (t1 to t5). At a reference numeral 242, a schematic diagram of the bulging region (S1 to S4; S1=0) for each elapsed time (t1 to t5) is shown. At a reference numeral 244, a graph is shown in which a vertical axis indicates the area of the bulging region (corresponding to S1 to S4 of reference numeral 242) and a horizontal axis indicates the elapsed time. The example shown in FIG. 19 shows the information on the change over time of the floating at the reference numerals 240, 242, and 244, but the present invention is not limited thereto. For example, the update unit 110H can acquire information on the change over time of a single or a plurality of pieces of floating, and can acquire information on a change over time of a type that is different from the information on the change over time of the floating shown in FIG. 19.

The update unit 110H acquires the information on the change over time of the floating, and updates the size of the flaked-off object that is estimated once. Specifically, the update unit 110H acquires an inspection result that is regularly performed as shown in FIG. 19, as the information on the change over time of the floating. The update unit 110H can estimate the size of the flaked-off object by, for example, analyzing the change over time between t1 and t3 to predict the size of the bulging region at t4 and/or t5. For example, in a case where the area of the bulging region at t3 is estimated to be S3, the update unit 110H acquires the information on the change over time of the floating shown in FIG. 19, and estimates and updates the bulging region at t4 to be S4.

Accordingly, in the present embodiment, it is possible to determine the risk degree of the flaked-off object corresponding to the change over time with high accuracy.

### Others

The building of the present embodiment is the tunnel, but the present invention is not limited thereto. Any building may be employed as long as the building is subjected to inspection, such as a bridge or a dam. The material of the surface of the building includes concrete repair materials such as reinforced concrete, concrete, and mortar.

Further, in the above embodiment, a hardware structure of a processing unit that executes various types of processing is the following various processors. The various processors include a central processing unit (CPU) which is a general-purpose processor that executes software (program) to function as various processing units, a programmable logic device (PLD) which is a processor whose circuit configuration can be changed after manufacturing such as a field programmable gate array (FPGA), a dedicated electric circuit which is a processor having a circuit configuration specifically designed to execute specific processing such as an application specific integrated circuit (ASIC), and the like.

One processing unit may be configured of one of these various processors or may be configured of two or more processors of the same type or different types (for example, a plurality of FPGAs or a combination of a CPU and an FPGA). Alternatively, a plurality of processing units may be composed of one processor. As an example of constituting the plurality of processing units by one processor, first, there is a form in which one processor is configured of a combination of one or more CPUs and software, as represented by a computer such as a client or a server, and the one processor functions as the plurality of processing units. Second, there is a form in which a processor that realizes the functions of the entire system including the plurality of processing units by one integrated circuit (IC) chip is used, as represented by a system on chip (SoC) or the like. As described above, various processing units are configured using one or more of the various processors as the hardware structure.

Further, the hardware structure of these various processors is more specifically an electric circuit (circuitry) in which circuit elements such as semiconductor elements are combined.

Each of the above configurations and functions can be realized by any hardware, software, or a combination of both, as appropriate. For example, the present invention can be also applied to a program causing a computer to execute the above processing step (processing procedure), a computer-readable recording medium (non-transitory recording medium) on which such a program is recorded, or a computer on which such a program can be installed.

Although the examples of the present invention have been described above, it is needless to say that the present invention is not limited to the embodiments described above and various modification examples can be made within a range not departing from the spirit of the present invention.

### EXPLANATION OF REFERENCES

10: three-dimensional measurement device
100: determination device
110: processor
110A: three-dimensional measurement data acquisition unit
110B: contour diagram generation unit
110C: bulging region detection unit
110D: size estimation unit
110E: weight estimation unit
110F: risk degree determination unit
110G: crack information acquisition unit
110H: update unit
120: memory
130: display
140: input/output interface
150: operation unit

What is claimed is:

1. A determination device that determines a predicted risk degree of a flaked-off object, the determination device comprising:
a processor; and
a memory that stores a program to be executed by the processor,
the processor being configured to:
acquire three-dimensional measurement data of a surface of a building measured by a measurement device;
detect a bulging region, which is a precursor of the flaked-off object, based on the three-dimensional measurement data;
estimate a size of the flaked-off object based on the bulging region;
estimate a weight of the flaked-off object from the size of the flaked-off object and a cover thickness from the surface of the building to a reinforcing bar or a steel material; and
determine the risk degree based on the weight.

2. The determination device according to claim 1,
the processor being configured to:
acquire information on a surface displacement from the three-dimensional measurement data; and
detect the bulging region based on the information on the surface displacement.

3. The determination device according to claim 1,
the processor being configured to:
acquire information on a surface displacement from the three-dimensional measurement data;
create a contour diagram of the surface based on the information on the surface displacement; and
detect a bulging of the surface based on the contour diagram to estimate the size of the flaked-off object based on the bulging of the surface.

4. The determination device according to claim 3,
the processor being configured to:
acquire crack information on the surface; and
correct the size of the flaked-off object based on the crack information and the contour diagram.

5. The determination device according to claim 4,
the processor being configured to:
acquire an image of the surface; and
acquire the crack information from the image.

6. The determination device according to claim 1,
the processor being configured to:
correct the determined risk degree based on the size of the flaked-off object.

7. The determination device according to claim 1,
the processor being configured to:
acquire information on a change over time of floating; and
update the size of the flaked-off object based on the information on the change over time of the floating.

8. The determination device according to claim 1,
the measurement device including a LiDAR or a stereo camera.

9. The determination device according to claim 8,
the three-dimensional measurement data being measured by the LiDAR of a frequency modulated continuous wave (FMCW) type.

10. The determination device according to claim 1,
a material of the surface of the building including concrete or a concrete repair material.

11. A determination method using a determination device that determines a predicted risk degree of a flaked-off object, the determination device including a processor and a memory that stores a program to be executed by the processor, the determination method comprising:
by the processor,
a step of acquiring three-dimensional measurement data of a surface of a building measured by a measurement device;
a step of detecting a bulging region, which is a precursor of the flaked-off object, based on the three-dimensional measurement data;
a step of estimating a size of the flaked-off object based on the bulging region;
a step of estimating a weight of the flaked-off object from the size of the flaked-off object and a cover thickness from the surface of the building to a reinforcing bar or a steel material; and
a step of determining the risk degree based on the weight of the flaked-off object.

12. A non-transitory computer readable medium storing a program that causes a computer to execute the determination method according to claim 11.
